(19) United States
(12) Patent Application Publication
(10) Pub. No.: US 2024/0013535 A1
(43) Pub. Date: Jan. 11, 2024

(54) IMAGE PROCESSING APPARATUS, IMAGE PROCESSING METHOD, AND RECORDING MEDIUM

(71) Applicant: OLYMPUS MEDICAL SYSTEMS CORP., Tokyo (JP)

(72) Inventors: Naoyuki MIYASHITA, Saitama (JP); Jun HANE, Tokyo (JP)

(73) Assignee: OLYMPUS MEDICAL SYSTEMS CORP., Tokyo (JP)

(21) Appl. No.: 17/858,212

(22) Filed: Jul. 6, 2022

(51) Int. Cl. G06V 20/40; G06T 7/50; G06T 7/60

(52) U.S. Cl. CPC G06V 20/41; G06T 7/50; G06T 7/60; G06V 20/46; G06T 2207/10016; G06T 2207/10068; G06T 2207/30004; G06T 2207/20084

(57) ABSTRACT

An image processing apparatus is provided with a processor. The processor is configured to receive a moving image acquired by an endoscope; classify a scene of the moving image; and determine observation coverage of the moving image using a determination method corresponding to the classified scene.

### TECHNICAL FIELD

The present invention relates to an image processing apparatus, an image processing method and a recording medium.

### BACKGROUND ART

In endoscopic examination, a scope is inserted into a lumen of an organ such as a stomach or a large intestine, and moving images are acquired and observed to check a lesion. Here, it is important to prevent a doctor from overlooking a lesion part.

One of causes of overlooking is that the doctor does not notice the lesion part existing in the image. In order to address the cause, a lesion detection technology like CADe/x (computer aided detection/diagnosis) is used to prevent overlooking by notifying the doctor of the existence of the lesion part.

Another cause of overlooking is that the lesion part does not exist in the image, and it is not possible to address this cause by the lesion detection technology. As a method for addressing this cause, a method of confirming observation coverage is proposed (see, for example, PTL 1 and NPL 1). PTL 1 discloses a technology of reconstructing a three-dimensional model of a luminal organ like a renal pelvis or a renal calyx from two-dimensional endoscopic images using a technology such as SLAM to make it possible to visually confirm an area the image of which has been already taken and an area the image of which has not been taken yet. NPL 1 discloses a technology of estimating a depth map, which is depth information about a large intestine, from endoscopic images, calculating a coverage rate of observation, and showing such an area of the large intestine that the cover rate is insufficient, in real time.

### CITATION LIST

### Patent Literature

{PTL 1} Japanese Examined Patent Application, Publication No. 6242543

### Non Patent Literature

{NPL 1} Daniel Freedman, et al, “Detecting deficient coverage in colonoscopies”, IEEE Transactions on Medical Imaging, Volume 39, issue 11, p. 3451-3462, 2020

### SUMMARY OF INVENTION

### Technical Problem

In order to reconstruct a three-dimensional model from images, it is necessary that a subject is stable and that the subject in the image is clear. In an actual observation flow of endoscopic examination, however, endoscopic images that enable reconstruction of a three-dimensional model and estimation of a depth map are not always acquired. Since the observation flow is considered in neither PTL 1 nor NPL 1, there is a possibility that observation coverage is not appropriately determined.

The present invention has been made in view of the above situation, and an object is to provide an image processing apparatus capable of appropriately determining observation coverage according to an observation flow during endoscopic examination, an image processing method and a recording medium.

### Solution to Problem

One aspect of the present invention is an image processing apparatus including a processor, wherein the processor is configured to receive a moving image acquired by an endoscope, classify a scene of the moving image and determine observation coverage of the moving image using a determination method corresponding to the classified scene.

Another aspect of the present invention is an image processing method including: receiving a moving image acquired by an endoscope, classifying a scene of the moving image and determining observation coverage of the moving image using a determination method corresponding to the classified scene.

Another aspect of the present invention is a non-transitory computer-readable recording medium recording an image processing program for causing a computer to execute: receiving a moving image acquired by an endoscope, classifying a scene of the moving image and determining observation coverage of the moving image using a determination method corresponding to the classified scene.

### Advantageous Effects of Invention

According to the present invention, there is an effect that it is possible to appropriately determine observation coverage according to an observation flow during endoscopic examination.

### BRIEF DESCRIPTION OF DRAWINGS

FIG. 1 is a configuration diagram of an image processing apparatus according to an embodiment.

FIG. 2 is a functional block diagram of a processor in the image processing apparatus.

FIG. 3A is a diagram describing wide observation.

FIG. 3B is a diagram showing an example of an endoscopic image in the wide observation.

FIG. 4A is a diagram describing close-up observation.

FIG. 4B is a diagram showing an example of an endoscopic image in the close-up observation.

FIG. 5A is a flowchart of an example of a first determination method.

FIG. 5B is a flowchart of another example of the first determination method.

FIG. 6A is a flowchart of an example of a second determination method.

FIG. 6B is a flowchart of another example of the second determination method.

FIG. 7 is a flowchart of an image processing method according to the embodiment.

FIG. 8 is a flowchart of a modification of the image processing method according to the embodiment.

### DESCRIPTION OF EMBODIMENTS

An image processing apparatus, an image processing method and an image processing program according to an embodiment of the present invention will be described with reference to drawings.

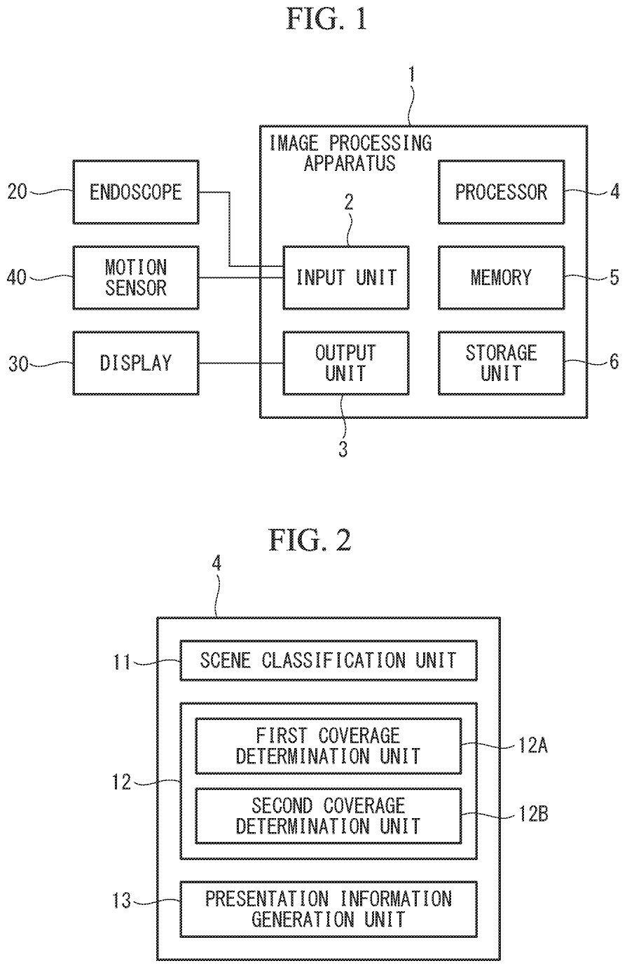

An image processing apparatus 1 according to the present embodiment has a function of processing a moving image acquired by an endoscope 20 and evaluating whether or not a subject has been comprehensively observed by the endoscope 20. As shown in FIG. 1, the image processing apparatus 1 may be a part of an endoscope system 10 provided with an endoscope 20 and a display 30.

The image processing apparatus 1 is provided with an input unit 2, an output unit 3, a processor 4, a memory 5 and a storage unit 6.

The input unit 2 has a publicly known input interface that is generally used to input a moving image. The moving image is composed of a plurality of time-series images. The input unit 2 is directly or indirectly connected to the endoscope 20, and a moving image acquired by the endoscope 20 is input into the image processing apparatus 1 through the input unit 2.

The output unit 3 has a publicly known output interface that is generally used to output a moving image. The output unit 3 is directly or indirectly connected to the display 30, and presentation information to be described later is output to the display 30 through the output unit 3. The output unit 3 may also output a moving image to the display 30.

The input unit 2 may be connected to peripheral equipment of the endoscope 20, and data acquired by the peripheral equipment may be input into the image processing apparatus 1 through the input unit 2. The peripheral equipment may be a motion sensor 40 that detects a motion of the tip of the endoscope 20, and sensor data of the motion sensor 40 may be input into the image processing apparatus 1 in synchronization with each image of the moving image. The motion sensor 40 is, for example, a shape detection device that detects an in-vivo shape of the endoscope 20 based on magnetism from the endoscope 20 or an IMU (inertial measurement unit) attached to the endoscope 20, and the sensor data includes data of a position and direction of the tip of the endoscope 20.

The processor 4 has at least one hardware like a central processing unit.

The memory 5 is a volatile memory like a RAM (random access memory) and functions as a work memory for the processor 4.

The storage unit 6 has a non-transitory computer-readable recording medium like a ROM (read-only memory) or a hard disk drive. The recording medium stores an image processing program for causing the processor 4 to execute an image processing method to be described later.

Next, a process executed by the processor 4 will be described.

As shown in FIG. 2, the processor 4 is provided with a scene classification unit 11, a coverage determination unit 12 and a presentation information generation unit 13 as functions. Functions of the units 11, 12 and 13 are realized by the processor 4 reading out the image processing program from the storage unit 6 to the memory 5 and executing a process according to the image processing program.

The scene of the moving image during endoscopic examination changes as time elapses according to an observation flow performed by a doctor.

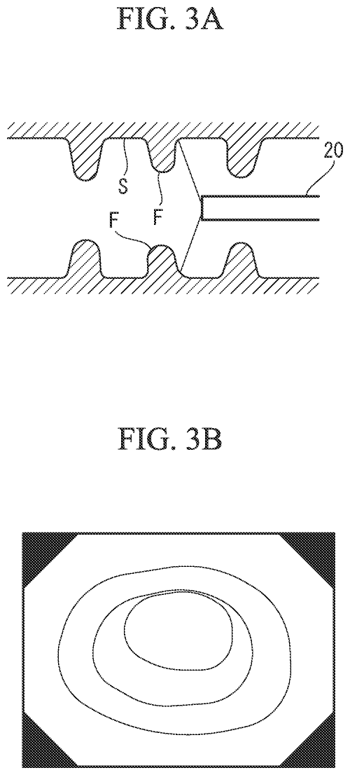

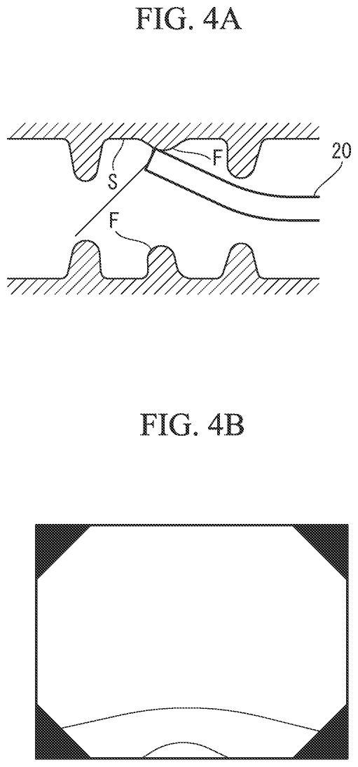

For example, in lesion screening of colonoscopy, the doctor inserts the endoscope 20 from the anus up to the cecum. After that, by pulling the endoscope 20, the doctor moves the observation range in the order of the cecum, the ascending colon, the transverse colon, the descending colon, the S-shaped colon and the rectum to observe the whole examination range. As shown in FIGS. 3A and 4A, many folds F exist in the intestines. By performing wide observation shown in FIG. 3A and, when necessary, close-up observation shown in FIG. 4A, the doctor observes the whole of each observation range without overlooking.

As shown in FIGS. 3A and 3B, in the scene of “wide observation”, the tip of the endoscope 20 is placed at a certain distance or longer from a subject S, such as an intestinal wall, and a wide range in the intestines is observed in a luminal direction. When the folds F are low, the back side of the folds F can be observed by the wide observation.

On the other hand, when the folds F are high, the back side of the folds F cannot be observed by the wide observation. When the back side of the folds F is not observed in the moving image, as shown in FIGS. 4A and 4B, the doctor deforms the folds F by pressing a vicinity of the tip of the endoscope 20 against the intestinal wall and causing the tip of the endoscope 20 to be close to the intestinal wall of the back side of the folds F, performs close-up observation of the intestinal wall. In the scene of “close-up observation”, a narrow range of the subject S is imaged from a short distance. The close-up observation includes clinical fold-back-side observation.

Thus, an observation range and observation conditions change in an observation flow as time elapses, and the scene of the moving image changes as time elapses.

The scene classification unit 11 classifies scenes of the moving image. The scenes of the moving image include three scenes: the “wide observation (a first scene)”, the “close-up observation (a second scene)” and the “others”. For example, the scene classification unit 11 classifies the scene of each of images constituting the moving image to any of the three scenes.

The “others” indicates a scene that is not suitable for determination of the observation coverage and includes “non-observation target”. The “non-observation target” indicates a scene in which the subject S, which is a target for observation by the doctor, is almost or completely not shown, or the subject S is unclear. For example, as the “non-observation target”, a so-called red ball scene that occurs by the endoscope being close to an intestinal wall and a scene in which objects other than the subject S, such as a residue, fed water and bubbles, are dominant are included.

As the “others”, “non-rigid object observation” may be included. The “non-rigid object observation” indicates a scene which includes, for example, a deformed non-rigid subject S such as winding of folds or a contracting organ, and from which a determination of observed and unobserved mucous membrane areas is difficult due to the presence of complicated conditions.

The scene classification unit 11 may classify scenes of the moving image using a publicly known image recognition technology based on a feature in the moving image. For the classification of scenes, an image classification method using deep leaning such as a CNN (convolutional neural network) may be used, or a classical classification method like an SVM (support vector machine) may be used.

The scene classification unit 11 may classify a scene of the moving image based on a subject distance from the tip of the endoscope 20 to a subject. In this case, a function of measuring the subject distance is provided. For example, the endoscope 20 may be a 3D endoscope 20 that acquires stereo images as time-series images constituting the moving image, or a distance sensor that measures a subject distance may be provided in the endoscope 20.

The coverage determination unit 12 judges whether the moving image is a target for determination of the observation coverage or not. Specifically, the coverage determination unit 12 judges that the moving image of any of the “wide observation” and the “close-up observation” is a target for determination and judges that the moving image of the “others” is not a target for determination.

The coverage determination unit 12 determines observation coverage of only the moving image that has been judged to be a target for determination, using a determination method corresponding to the scene of the moving image. The observation coverage means that the whole of a subject which is an observation target is observed by the endoscope 20 without overlooking. In other words, the coverage determination unit 12 determines or detects an unobserved area that has not been observed by the endoscope 20, that is, an area overlooked by the doctor.

The coverage determination unit 12 is provided with a first coverage determination unit 12A for the “wide observation” and a second coverage determination unit 12B for the “close-up observation”.

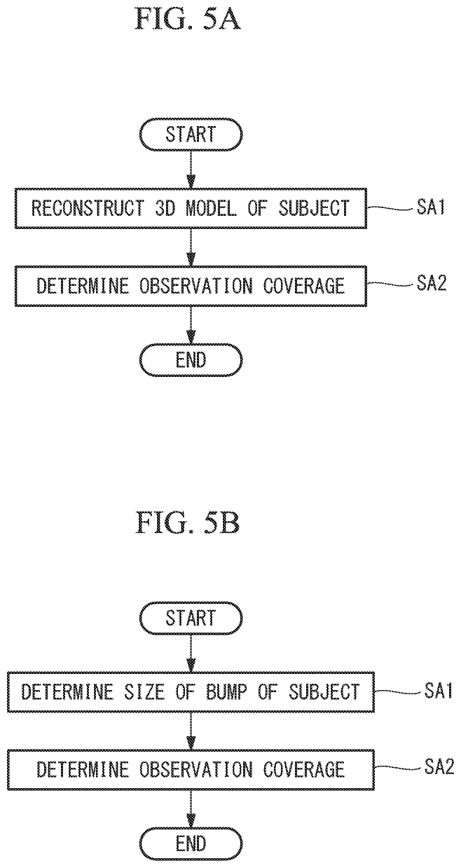

The first coverage determination unit 12A determines observation coverage of a moving image of the “wide observation” using a first determination method. FIGS. 5A and 5B show specific examples of the first determination method. As shown in FIGS. 5A and 5B, the first determination method includes step SA1 of estimating a solid shape of a subject and step SA2 of determining observation coverage based on the estimated solid shape of the subject.

In the first determination method of FIG. 5A, the first coverage determination unit 12A reconstructs a three-dimensional (3D) model of the subject from the moving image (step SA1) and determines observation coverage based on the reconstructed 3D model (step SA2). In the reconstruction of the 3D model, sensor data of the motion sensor 40 may be used as necessary. The 3D model can be reconstructed using a publicly known visual SLAM method. In the case of using the sensor data together, the 3D model is reconstructed using the publicly known visual-inertial SLAM method.

At step SA1, a 3D model of an observed area included in the moving image is reconstructed, and a 3D model of an unobserved area not included in the moving image is not reconstructed. At step SA2, the first coverage determination unit 12A may determine the observation coverage based on whether there is a missing area of the reconstructed 3D model. Further, the observation coverage may be indexed based on the area of the missing area of the 3D model.

In the first determination method of FIG. 5B, the first coverage determination unit 12A determines the size of a bump on the surface of the subject from the moving image (step SA1) and determines observation coverage based on the size of the bump (step SA2).

When a bump like a fold F is small (low), the back side of the bump is also observed in the moving image. On the other hand, when the bump like a fold F is large (high), the back side of the bump is not observed in the moving image. At step SA2, the first coverage determination unit 12A may determine that there is no unobserved area if the size of the bump is equal to or smaller than a predetermined value and determine that there is an unobserved area if the size of the bump is larger than the predetermined value.

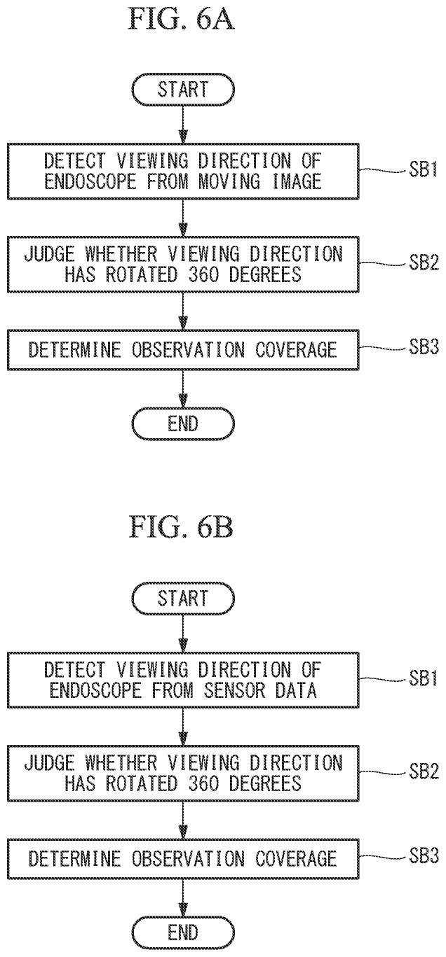

The second coverage determination unit 12B determines observation coverage of the moving image of the “close-up observation” using a second determination method. FIGS. 6A and 6B show specific examples of the second determination method. As shown in FIGS. 6A and 6B, the second determination method includes step SB1 of estimating a viewing direction of the endoscope 20 and steps SB2, SB3 of determining observation coverage based on the estimated viewing direction.

In the “close-up observation”, the doctor causes the tip of the endoscope 20 to make one turn like drawing a circle by causing the bending direction of a bending portion of the endoscope 20 to change, and, thereby, causes the viewing direction of the endoscope 20 to rotate 360 degrees in the circumferential direction. Therefore, it is possible to determine observation coverage based on the viewing direction of the endoscope 20.

In the second determination method of FIG. 6A, the second coverage determination unit 12B detects the viewing direction of the endoscope 20 from time-series images constituting the moving image (step SB1), determines whether the viewing direction has rotated 360 degrees or not (step SB2) and determines observation coverage based on whether the viewing direction has rotated 360 degrees or not (step SB3). As a method for detecting the viewing direction from the time-series images, for example, a publicly known relative camera pose estimation method such as a five-point algorithm using RANSAC (random sample consensus) or a method of simply calculating the viewing direction from a locus of time-series motion vectors is used.

In the second determination method of FIG. 6B, the second coverage determination unit 12B detects the viewing direction of the endoscope 20 from sensor data using the motion sensor 40 (step SB1), determines whether the viewing direction has rotated 360 degrees or not (step SB2) and determines observation coverage based on whether the viewing direction has rotated 360 degrees or not (step SB3).

At step SB3, the second coverage determination unit 12B may determine that there is no unobserved area if the viewing direction has rotated 360 degrees and determine that there is an unobserved area if the viewing direction has not rotated 360 degrees.

As for coverage of the viewing direction, it is possible to judge whether one rotation has been made or not by calculating an angle rate of observation relative to 360 degrees from time-series changes in the viewing direction and comparing the angle rate with a predetermined threshold.

The presentation information generation unit 13 generates presentation information based on a result of the determination of the coverage determination unit 12 and sequentially outputs the presentation information to the display 30. By the presentation information being displayed on the display 30, a result of observation coverage determination is presented to the doctor in real time.

The presentation information may include information about whether the moving image is a target for determination of the observation coverage or not (that is, whether determination or detection of the observation coverage has been executed or not). For example, if the moving image is not a target for determination, the presentation information may include an alert display. The observer can recognize whether detection of an unobserved area is executed by the image processing apparatus 1 or not, based on the presentation information displayed on the display 30.

If the moving image is a target for determination, the presentation information may include information about a result of observation coverage determination, for example, information about presence/absence of an unobserved area and information about a position and direction of the unobserved area. In one example, the presentation information may include the reconstructed 3D model. The presentation information may include the angle rate of the viewing direction.

The presentation information displayed on the display 30 may be selectable by the doctor.

Next, an image processing method executed by the processor 4 will be described.

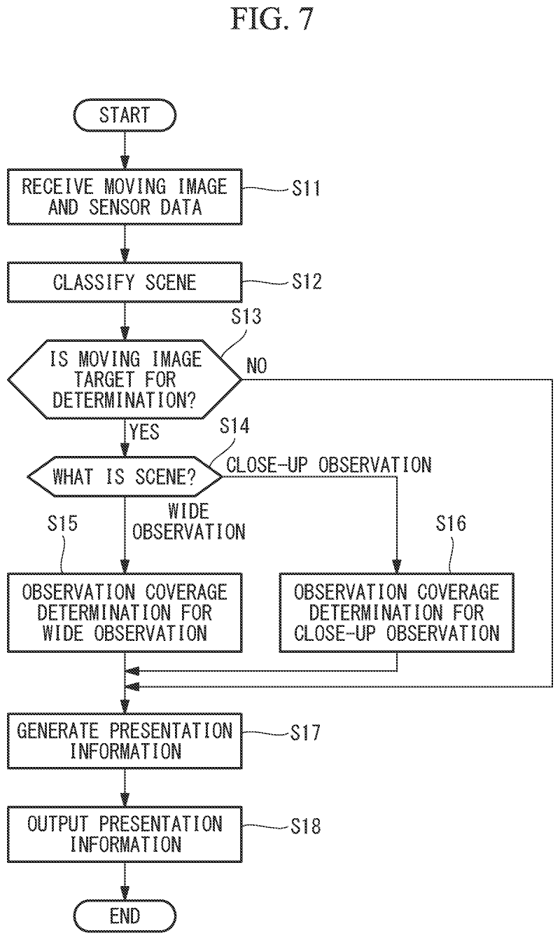

As shown in FIG. 7, the image processing method according to the present embodiment includes step S11 of receiving a moving image, step S12 of classifying the scene of the moving image, step S13 of judging whether the moving image is a target for determination of observation coverage or not, steps S14 to S16 of determining observation coverage of the moving image using a determination method corresponding to the scene of the moving image, step S17 of generating presentation information and step S18 of outputting the presentation information.

First, the processor 4 receives a moving image input into the image processing apparatus 1 (step S11). At step S11, the processor 4 may receive sensor data detected by the motion sensor 40 as necessary.

Next, the scene of the moving image is classified to any of the three scenes by the scene classification unit 11 (step S12).

Then, whether the moving image is a target for determination or not is judged by the scene classification unit 11 based on the scene classified at step S12 (step S13). Specifically, if the scene is the “wide observation” or the “close-up observation”, the moving image is judged to be a target for determination. If the scene is the “others”, the moving image is judged not to be a target for determination.

If the moving image is judged to be a target for determination (step S13: YES), then observation coverage of the moving image is determined by the coverage determination unit 12 using a determination method corresponding to the scene (steps S14 to S16).

Specifically, it is judged whether the scene is the “wide observation” or the “close-up observation” (step S14).

If the scene is the “wide observation”, then the observation coverage of the moving image is determined by the first coverage determination unit 12A (step S15). At step S15, the first determination method for the “wide observation” shown in FIG. 5A or 5B is used.

If the scene is the “close-up observation”, then the observation coverage of the moving image is determined by the second coverage determination unit 12B (step S16). At step S16, the second determination method for the “close-up observation” shown in FIG. 6A or 6B is used.

Next, presentation information based on results of the judgments and determinations of steps S13 to S16 is generated by the presentation information generation unit 13 (step S17). The generated presentation information is output from the processor 4 to the display 30 and displayed on the display 30.

Thus, according to the present embodiment, the scene of a moving image is classified, and a determination method used for determination of observation coverage of the moving image is changed according to the scene. Thereby, it is possible to appropriately determine observation coverage using a determination method suitable for an observation flow performed by a doctor, and effectively support comprehensive observation in actual endoscopic examination.

Specifically, factors in overlooking differ according to scenes, and, therefore, measures against overlooking differ according to the scenes.

One of factors causing overlooking in the “wide observation” is non-observation of the back side of a bump like a fold F. In this case, it is possible to determine observation coverage with a high reliability based on a 3D model estimated from the moving image or a solid shape of the subject, such as the size of the bump.

Another factor causing overlooking in the “wide observation” is that viewing directions are biased. In this case, it is possible to determine observation coverage with a high reliability based on a reconstructed 3D model of the subject.

One of factors causing overlooking in the “close-up observation” is that one rotation of the viewing direction has not been made. Further, though it is necessary that a subject in images are clear in order to reconstruct a 3D model from the image using a technology like SLAM, the moving image of the “close-up observation” is not suitable for reconstruction of a 3D model because defocus or blur of the subject easily occurs. Therefore, in the case of the “close-up observation”, it is possible to determine observation coverage with a high reliability by judging whether one rotation of the viewing direction of the endoscope 20 has been made or not.

Further, according to the present embodiment, whether the moving image is a target for determination of observation coverage or not is judged, and observation coverage determination of the moving image that is not the target for determination is not performed. Thereby, it is possible to prevent wrong determination of the observation coverage and present presentation information with a high reliability that is effective for support to an observer.

For example, in the case of the “non-rigid object observation”, it is difficult to estimate an accurate solid shape of the subject by reconstruction of a 3D model or the like because there are complicated conditions such as that the subject deforms as time elapses. By excluding such a scene from which observation coverage determination is technologically difficult, from target for determinations, it is possible to effectively prevent wrong determination.

“Non-observation targets” indicates a scene that is not suitable for examination. By excluding such a scene from target for determinations, it is possible to prevent presentation information unnecessary for the doctor from being presented.

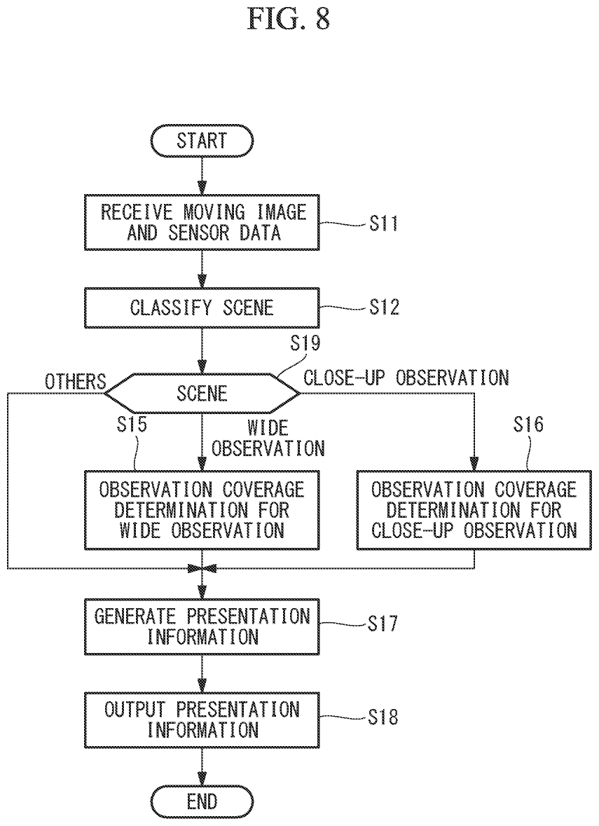

In the above embodiment, it is assumed that judgment of a target for determination and selection of a determination method are executed at two steps S12 and S13. Instead, the judgment and the selection may be executed by one step S19 as shown in FIG. 8.

That is, after the scene is classified at step S12, the next step is selected according to the scene at step S19. Specifically, step S15 is executed next if the scene is the “wide observation”; step S16 is executed next if the scene is the “close-up observation”; and step S17 is executed next without steps S15 and S16 being executed if the scene is the “others”.

In the above embodiment, it is assumed that the first scene is the “wide observation” in which a subject is imaged from a long distance. However, the first scene is not limited thereto, and may be other scenes from which a solid shape of the subject can be estimated. That is, the first scene is a scene with a large amount of information about the structure of a subject, which is required to estimate a solid shape, and may be an arbitrary scene with a wide field of view including an uneven structure of the subject like the folds F, in which the subject is clearly imaged. Using characteristics of an acquired image, the first scene is defined as a scene an image of which includes a predetermined or larger amount of information such as contrast and texture strength, due to a mucous membrane structure of folds, a bump or the like and blood vessels below mucous membranes.

In the above embodiment, it is assumed that the second scene is the “close-up observation” in which a subject is imaged from a short distance. However, the second scene is not limited thereto, and may be other scenes from which it is difficult to estimate a solid shape of the subject and in which the subject is observed by changing the viewing direction of the endoscope 20. That is, the second scene is a scene an image of which includes a small amount of information about the structure of the subject. Using characteristics of an acquired image, the second scene is defined as a scene with less information about the image due to defocus caused by the endoscope being close to mucous membranes or blur caused by the motion of the endoscope and the like, compared to the first scene.

Further, “the first scene”, “the second scene” and the “others” are subdivided scene classifications or scene classifications with some overlapping conditions, and observation coverage determination according to the classifications may be performed.

In the above embodiment, it is assumed that the presentation information is displayed on the display 30 in real time. However, at least a part of the presentation information may be displayed on the display 30 after the endoscopic examination. The doctor can check whether there is overlooking or not based on the presentation information after the endoscopic examination.

In the above embodiment, it is assumed that all the processes from steps S12 to S17 are executed by the processor 4 inside the image processing apparatus 1. Instead, at least a part of the processes from steps S12 to S17 may be executed by an arbitrary apparatus outside the image processing apparatus 1. For example, the image processing apparatus 1 may be connected to a cloud server via a communication network, and the classification of the scene at step S12 may be executed by the cloud server.

In the above embodiment, description has been made on the image processing apparatus 1 that processes an image output from the endoscope 20. However, the image processing apparatus of the present invention can be applied to medical equipment including an endoscope, a medical manipulator and equipment obtained by electrification/automation thereof. Further, the image processing apparatus may be implemented as an arbitrary analysis apparatus that processes or analyzes an image output from the medical equipment or may be implemented as medical equipment to which the functions described above are added.

The embodiment of the present invention has been described above in detail with reference to drawings. A specific configuration, however, is not limited to the above embodiment, and design changes and the like within a range not departing from the scope of the present invention are also included. Further, the components shown in the above embodiment and modifications can be appropriately combined and configured.

### REFERENCE SIGNS LIST

1 Image processing apparatus
4 Processor
6 Storage unit (recording medium)
S Subject

1. An image processing apparatus comprising a processor, wherein
the processor is configured to:
receive a moving image acquired by an endoscope;
classify a scene of the moving image; and
determine observation coverage of the moving image using a determination method corresponding to the classified scene.

2. The image processing apparatus according to claim 2, wherein
the processor is further configured to judge whether the moving image is a target for determination of the observation coverage or not; and
the processor determines the observation coverage of only the moving image that has been judged to be the target for determination.

3. The image processing apparatus according to claim 2, wherein
the processor classifies the scene of the moving image to any of a plurality of scenes, the plurality of scenes including a first scene with a large amount of information about a structure of a subject and a second scene with a small amount of information about the structure of the subject;
the target for determination includes a moving image of the first scene and a moving image of the second scene; and
the processor uses a first determination method for determination of the observation coverage of the moving image of the first scene and uses a second determination method different from the first determination method for determination of the observation coverage of the moving image of the second scene.

4. The image processing apparatus according to claim 2, wherein
the first determination method comprises:
estimating a solid shape of the subject from the moving image; and
determining the observation coverage based on the solid shape of the subject.

5. The image processing apparatus according to claim 4, wherein
the estimating of the solid shape comprises reconstructing a three-dimensional model of the subject from the moving image; and
the determining of the observation coverage is performed based on the three-dimensional model.

6. The image processing apparatus according to claim 4, wherein
the estimating of the solid shape comprises determining a size of a bump on a surface of the subject from the moving image; and
the determining of the observation coverage is performed based on the size of the bump.

7. The image processing apparatus according to claim 2, wherein
the second determination method comprises:
estimating a viewing direction of the endoscope; and
determining the observation coverage based on the viewing direction of the endoscope.

8. The image processing apparatus according to claim 7, wherein
the estimating of the viewing direction comprises detecting the viewing direction of the endoscope from the moving image; and
the determining of the observation coverage is performed by judging whether or not the viewing direction has rotated 360 degrees in a circumferential direction.

9. The image processing apparatus according to claim 7, wherein
the estimating of the viewing direction comprises detecting the viewing direction of the endoscope using a motion sensor; and
the determining of the observation coverage is performed by judging whether or not the viewing direction has rotated 360 degrees in the circumferential direction.

10. The image processing apparatus according to claim 1, wherein
the processor classifies the scene of the moving image based on a feature in the moving image.

11. The image processing apparatus according to claim 1, wherein
the processor classifies the scene of the moving image based on a subject distance.

12. An image processing method comprising:
receiving a moving image acquired by an endoscope;
classifying a scene of the moving image; and
determining observation coverage of the moving image using a determination method corresponding to the classified scene.

13. A non-transitory computer-readable recording medium recording an image processing program for causing a computer to execute:
receiving a moving image acquired by an endoscope;
classifying a scene of the moving image; and
determining observation coverage of the moving image using a determination method corresponding to the classified scene.
